(19) United States
(12) Patent Application Publication
(10) Pub. No.: US 2025/0017456 A1
(43) Pub. Date: Jan. 16, 2025

(54) ENDOSCOPIC CONTROL APPARATUS, ENDOSCOPE SYSTEM, MEDICAL SYSTEM, AND ENDOSCOPIC CONTROL METHOD

(71) Applicant: OLYMPUS MEDICAL SYSTEMS CORP., Tokyo (JP)

(72) Inventors: Osamu NONAKA, Sagamihara-shi (JP); Masahiro ASHIZUKA, Tokyo (JP)

(73) Assignee: OLYMPUS MEDICAL SYSTEMS CORP., Tokyo (JP)

(21) Appl. No.: 18/765,513

(22) Filed: Jul. 8, 2024

Related U.S. Application Data
(60) Provisional application No. 63/525,727, filed on Jul. 10, 2023.

(51) Int. Cl. A61B 1/045; A61B 1/00

(52) U.S. Cl. CPC A61B 1/045; A61B 1/000094; A61B 1/0005

(57) ABSTRACT

An endoscopic control apparatus includes one or more processors configured to process image matching with respect to a first image and a second image to determine an image in the first image and the second image, the first image obtained from an endoscope during an endoscopy, the image feature comprises information related to common pixels among the first image and the second image, detect a treatment target based on the image feature obtained from the image matching, determine a treatment instrument based on the detected treatment target, and display guide information, the guide information includes information related to the treatment instrument.

### RELATED APPLICATION DATA

This application is based on and claims priority under 35 U.S.C. § 119 to U.S. Provisional Application No. 63/525,727, filed on Jul. 10, 2023, the entire contents of which are incorporated herein by reference.

### BACKGROUND

The present disclosure relates to a display control apparatus for endoscopy, a display control method for endoscopy, a non-transitory recording medium recording display control program for endoscopy, a medical system, and a training data creation apparatus, with which an appropriate advice can be provided to a doctor or medical personnel upon inspection or treatment using an endoscope or preparation for the inspection or treatment.

### DESCRIPTION OF RELATED ART

An endoscope is a device inserted to an inside of a body or the like with which an affected part or the like which cannot be seen from the outside can be observed. Upon observation of the inside of the body or the like, pretreatment or anesthesia can be administered to an examinee. In endoscopy, a hygienic state also can be managed. In the endoscopy, to reduce burden of an operator, the examinee, and an assistant, a smooth inspection or treatment is demanded in an environment with many constraints.

### SUMMARY

An endoscopic control apparatus includes one or more processors configured to process image matching with respect to a first image and a second image to determine an image in the first image and the second image, the first image obtained from an endoscope during an endoscopy, the image feature comprises information related to common pixels among the first image and the second image, detect a treatment target based on the image feature obtained from the image matching, determine a treatment instrument based on the detected treatment target, and display guide information, the guide information includes information related to the treatment instrument.

An endoscopic control method according to an aspect of the present disclosure includes processing image matching with respect to a first image and a second image to determine an image feature in the first image and the second image, the first image obtained from an endoscope during an endoscopy, the image feature comprises information related to common pixels among the first image and the second image, detecting a treatment target of the image feature obtained from the image matching, determining a treatment instrument based on the image feature of the treatment target, and displaying guide information, the guide information includes information related to the treatment instrument.

A medical system according to an aspect of the present disclosure includes an endoscope, display equipment, and an endoscopic control apparatus including a processor, the processor configured to detect a treatment target of an image feature based on a picked-up image obtained from an endoscope during endoscopy, determines a treatment instrument based on the image feature of the treatment candidate, and generate a guide information, the guide information includes information related to the treatment instrument.

An endoscopic control method according to another aspect of the present disclosure includes detecting a treatment target of image feature based on a picked-up image obtained from an endoscope during endoscopy, identifying a treatment instrument based on the image feature of the treatment target, generating guide information in a guide display. Identifying the treatment instrument based on the image feature of the treatment target includes detecting frames from among continuous frames from a plurality of movies obtained by the endoscope during endoscopy and in which a treatment instrument appears, and determining the treatment instrument using an inference model applied to the detected frames. The inference model is obtained from learning training data obtained by annotating a treatment instrument appearing in a series of frames of an endoscopic image obtained from the endoscope during the endoscopy. The inference model correlates a relationship between a treated treatment target image and the treatment instrument used. The guide information includes information related to the identified treatment instrument.

An endoscopic control method, comprises detecting a treatment target of a first image feature from first images obtained from an image pick-up device of an endoscope during a first endoscopy, determine a second image feature based on the first image feature, the second image feature is obtained from a second images during a second endoscopy being conducted prior to the first endoscopy, determining a treatment instrument based on the second image feature, the treatment instrument being included in third image subsequent to the second image, and determining a treatment instrument candidate corresponding to the treatment instrument, and displaying guide information including information related to the treatment instrument candidate.

### BRIEF DESCRIPTION OF THE DRAWINGS

FIG. 1 is a block diagram illustrating a medical action preparation guide apparatus according to a first embodiment.

FIG. 2 is an explanatory diagram for describing arrangement information upon endoscopy.

FIG. 3 is an explanatory diagram illustrating a distal end portion of an insertion portion 21.

FIG. 4 is an explanatory diagram illustrating an example of a treatment instrument.

FIG. 5 is an explanatory diagram illustrating a state in which a treatment instrument TT is placed on a cart CA.

FIG. 6 is an explanatory diagram illustrating a situation of the endoscopy.

FIG. 7 is an explanatory diagram for describing a treatment on a treatment candidate target.

FIG. 8 is a flowchart for describing an outline of the treatment in the endoscopy.

FIG. 9 is a flowchart for describing operation in the endoscopy.

FIG. 10 is an explanatory diagram for describing a generation method of a treatment instrument inference model.

FIG. 11 is an explanatory diagram for describing the generation method of the treatment instrument inference model.

FIG. 12 is a flowchart illustrating a creation (learning) method of the treatment instrument inference model.

FIG. 13 is a block diagram illustrating a display control apparatus for endoscopy according to a second embodiment.

FIG. 14 is an explanatory diagram illustrating an example of information registered in treatment instrument DB 10.

FIG. 15 is a block diagram illustrating a third embodiment.

FIG. 16 is a flowchart for describing an operation of the third embodiment.

### DETAILED DESCRIPTION

In endoscopy, a treatment may be performed on a lesion part in a middle of an inspection. A doctor selects a treatment instrument suitable to the treatment according to a type, a size, or the like of the lesion part. However, unless the doctor is an expert, it may not be easy to appropriately select an optimal treatment instrument from among a wide variety of treatment instruments. For example, in a middle of the endoscopy, when a polyp, a foreign body, a parasite, and the like are found, depending on an experience of the doctor, it may not be easy to determine a treatment instrument that is to be selected as a treatment instrument which treats the polyp, the foreign body, the parasite, and the like. To smoothly implement the endoscopy, it is useful to provide an appropriate advice to the doctor on a selection or the like of the treatment instrument.

Note that the advice to the doctor can be provided by an image, a voice, various indicators, and the like. Note that in the present embodiment, an example will be described in which the advice is provided to the doctor by a display configured to display an image, but a method of providing the advice is not particularly limited.

As such a display, any form may be adopted such as a stationary type, a handheld type, or a wearable type. As a display control apparatus (endoscopic control apparatus) for endoscopy configured to supply information for some advice (hereinafter, referred to as guide information) to the display, not only a standalone apparatus but also an apparatus on cloud may be used. Furthermore, the guide information may be provided to the display by IoT-like solutions. A location where the display control apparatus for endoscopy is arranged can be freely set.

For example, the display control apparatus for endoscopy may be provided on a cart for transporting an endoscope from a storage to an inspection room, and in this case, the cart can be used as a smart cart having an intelligent function. Such a smart cart can be set to be communicable with medical equipment such as the treatment instrument which is placed on a cart, so that a status of management, transport, and the like of the prepared medical equipment can be grasped, and it is possible to address various issues for providing the guide information.

The smart cart can be a cart for transporting equipment comprising a computer or a processor configured to communicate with one or more of other computers and/or cloud computing so as to obtain information and assist user (or operator) based on the predetermined program. The user can control the assist of the smart cart by the instruction via the input device such as a keyboard, a touch panel, a switch, or a button. The smart cart can communicate a processor of the endoscope by wired and wireless manner, such as LAN, WAN, Bluetooth™ or wireless-fidelity (Wi-Fi) direct. The smart cart can be easily moved by wireless communication. The smart cart can be controlled based on the situation of the subject (patient) detected by the medical device such as the endoscope. The smart can also communicate with the treatment instrument on the smart cart. Therefore, the user can grasp information related to a location of the treatment instrument and a status of the transportation of the treatment instrument. This information can be used for the guide information. The information can be provided to the user through device such as the display equipped on the smart cart, the speaker equipped on the smart cart, other display, the speaker or the wearable device of the user. These display, speaker, wearable device can transmit the information to other devices. By providing one or more of a barcode, matrix barcode, IC tag or image recognition, an image sensor, the smart cart can determine whether the treatment instrument on the smart cart, where the treatment instrument on the smart cart is, whether the treatment instrument is used. When the image sensor is provided, the smart cart can determine the treatment tool based on the shape of color of the treatment instrument. And such determining can be conducted when the user picks up the treatment instrument and/or uses the treatment instrument during the endoscopy. The smart cart can also comprise a sensor that can detects one or more accelerate motion and location. To obtain the location of the smart cart, the smart cart can communicate with gates or cameras in medical facilities. The smart cart can move individually and automatically by motor or actuator.

Note that Japanese Patent Application Laid-Open Publication No. 2021-74243 discloses a technology for assisting a medical action by an apparatus configured to estimate an instrument being used based on a motion of a surgeon. However, in this proposal, a plurality of dynamic sensors mounted to the surgeon, a camera configured to pick up an image of a behavior of the surgeon and an affected part, and the like need to be provided, and a system is on a large scale.

Hereinafter, embodiments of the present disclosure will be described in detail with reference to the drawings.

### First Embodiment

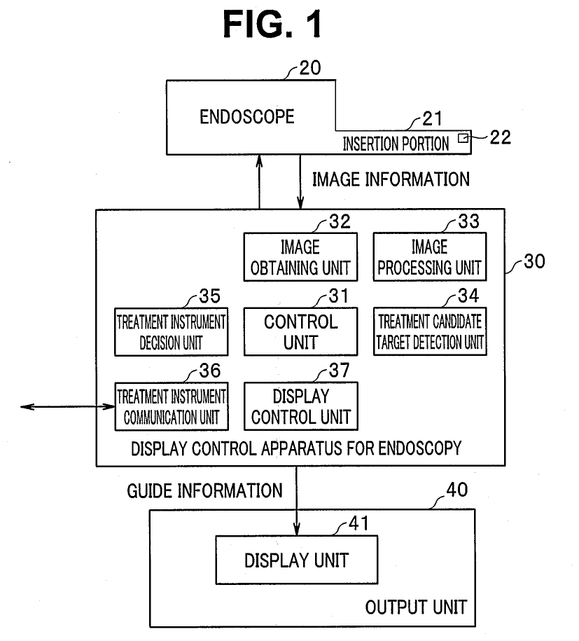

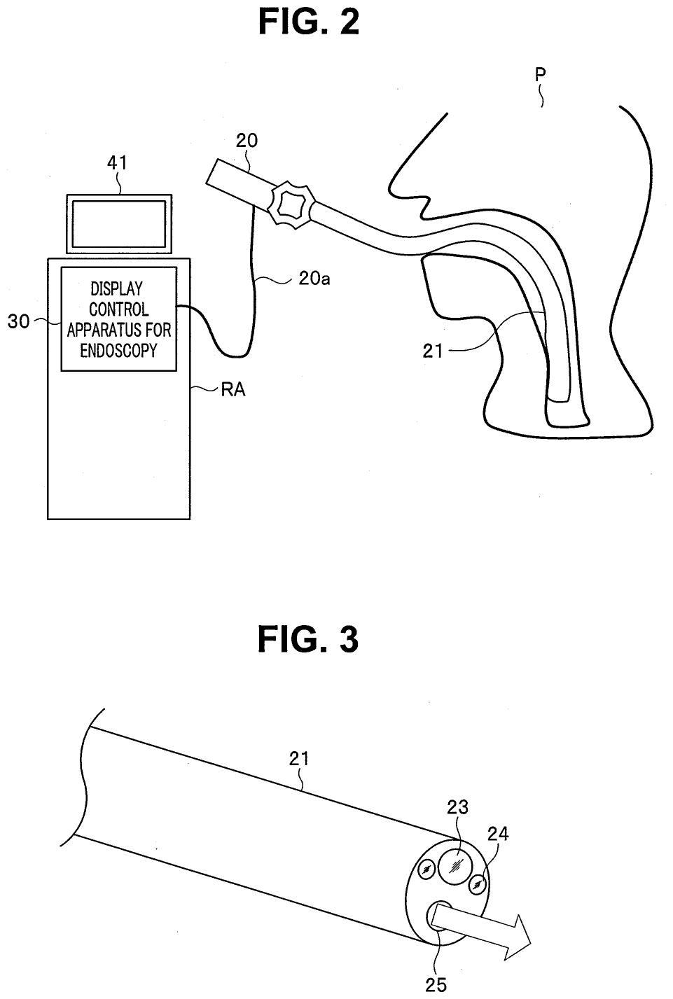

FIG. 1 is a block diagram illustrating a display control apparatus for endoscopy according to a first embodiment. FIG. 2 is an explanatory diagram illustrating an external appearance of a medical system including the display control apparatus for endoscopy. The present embodiment is intended to effectively assist a medical action such as an inspection or a treatment by detecting a treatment candidate target (treatment target) such as a lesion part based on an image obtained by the inspection, and determining a candidate of the treatment instrument suitable to the treatment on the treatment candidate target to present the candidate to the doctor or the like.

FIG. 2 illustrates a display control apparatus for endoscopy 30 and a display unit 41 which are placed on a stand RA. An endoscope 20 is connected to the display control apparatus for endoscopy 30 by a cable 20a. The endoscope 20 has an elongated insertion portion 21 with flexibility which is inserted to a body cavity of a patient P, for example.

The display unit 41 configured to display the guide information can be provided in a location that can be visually recognized by the doctor or the like who operates the endoscope 20. The example in FIG. 2 illustrates the example in which the display control apparatus for endoscopy 30 and the display unit 41 are placed on the stand RA, but the display control apparatus for endoscopy 30 may be provided in any location as long as image information from the endoscope 20 can be received, and display data of the guide information can be supplied to the display unit 41.

In FIG. 1, the image information from the endoscope 20 is inputted to the display control apparatus for endoscopy 30. The endoscope 20 includes an image pickup device 22. The image pickup device 22 is provided, for example, at a distal end of the insertion portion 21. The endoscope 20 has an optical system which is not illustrated and which is configured to guide an optical image of a subject to an image pickup surface of the image pickup device 22. The image pickup device 22 is configured by a CCD, a CMOS sensor, or the like, and configured to photoelectrically convert the optical image of the subject from the optical system to obtain a picked-up image (image pickup signal) of the subject. Note that the optical system includes lenses, an aperture, and the like, which are not illustrated, for zooming and focusing. The optical system may include a zoom (variable magnification) mechanism and a focus and aperture mechanism which are not illustrated and which are configured to drive these lenses. The image information of the picked-up image (endoscopic image) obtained by preforming image pickup by the image pickup device 22 is supplied to the display control apparatus for endoscopy 30.

An insertion slot which is not illustrated may be provided in the endoscope 20. In this case, an operator can insert the treatment instrument from the insertion slot to cause the treatment instrument to protrude from a distal end opening of an insertion portion, so that the treatment can be performed.

FIG. 3 is an explanatory diagram illustrating a distal end portion of the insertion portion 21. A large number of holes are provided in the insertion portion 21 in an axial direction of the insertion portion 21, and various internal components are fixed to these holes. For example, an objective optical system which is not illustrated is arranged so as to face an observation window 23 provided in a distal end surface of the insertion portion 21, and an image pickup surface of the image pickup device 22 is arranged in an image forming location of the objective optical system. An illumination window 24 for emitting illumination light towards the subject is also provided in the distal end surface of the insertion portion 21.

An insertion slot, which is not illustrated, for the treatment instrument such as forceps is provided on a proximal end side of the endoscope 20, and the treatment instrument inserted from the insertion slot is configured to be able to be inserted into and through a channel provided in a longitudinal direction of the insertion portion 21. Furthermore, the channel communicates with an opening portion 25 on the distal end surface of the insertion portion 21, and the treatment instrument inserted from the insertion slot can protrude in an arrow direction of FIG. 3 from the opening portion 25.

Various instruments necessary to discover and treat the polyp, the foreign body, the parasite, and the like are proposed as the treatment instruments. For example, the treatment instruments include a snare which is made of a looped wire and which is configured to remove the polyp, biopsy forceps which can pinch and collect part of tissue, grasping forceps configured to collect resected tissue, basket type grasping forceps of a basket type made of a wire, a high frequency knife which cuts out a lesion by applying a high frequency current, and a syringe needle for injecting a drug or the like. Even when the treatment instruments are of a same type, a wide variety of treatment instruments having different distal end shapes, treatment instruments having different sizes, and the like exist. Experiences and knowledge are required to quickly select a suitable treatment instrument from the wide variety of treatment instruments described above, and depending on a situation, it may take time and trouble.

Such a treatment instrument may be placed on the cart arranged in an inspection room, or may be stored in a hospital. For some treatment instruments, it may be necessary to place an order with a manufacturer or the like. For example, an electronic tag such as a RFID (radio frequency identification) is attached to the treatment instrument, and the electronic tag is caused to store information related to the treatment instrument, so that it is possible to obtain information related to each treatment instrument by communication. Accordingly, for example, it is also possible to grasp a treatment instrument existing in a close location, such as a treatment instrument placed on the cart in the inspection room. With such devices, efficiency can be achieved in various ways by quickly grasping treatment instruments at hand and immediately arranging treatment instruments that are not at hand or the like, so that time loss can be reduced. The guide information includes a location of the treatment instrument.

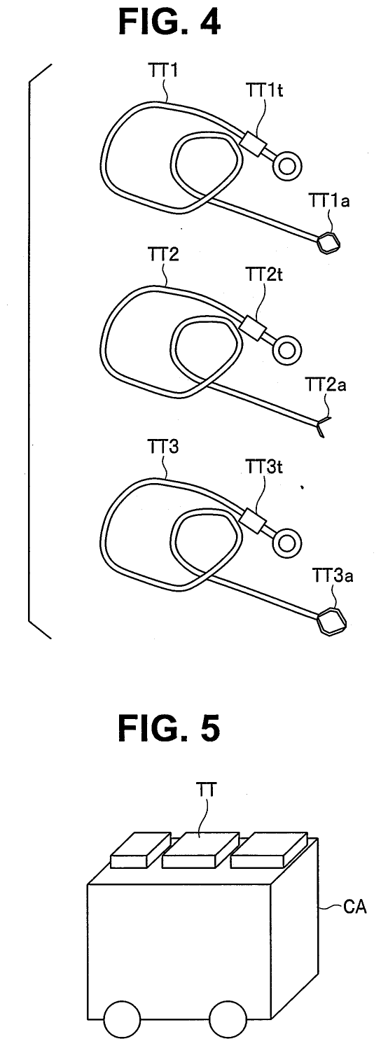

FIG. 4 is an explanatory diagram illustrating an example of the treatment instrument. FIG. 4 illustrates three treatment instruments TT1 to TT3 (hereinafter, which are referred to as a treatment instrument TT when there is no need to distinguish between the treatment instruments). The treatment instrument TT1 is a snare in which a distal end portion TT1a is made of a looped wire. The treatment instrument TT2 is biopsy forceps configured to pinch tissue by a distal end portion TT2a. The treatment instrument TT3 is a snare having a loop in a distal end portion TT3a which is larger than a loop of the treatment instrument TT1.

Herein, TT1, TT2, and TT3 respectively have electronic tags TT1t, TT2t, and TT3t (hereinafter, which are referred to as an electronic tag TTt when there is no need to distinguish between the electronic tags). The electronic tags TT1t to TT3t hold information respectively indicating what types of treatment instruments the treatment instruments TT1 to TT3 are. Each of the electronic tags TTt that is a proximity wireless communication device is configured by the RFID, for example, and can transmit the held information to a communication device in a close location. It goes without saying that communication is not limited to such wireless communication, and determination may be made by using a wired connection. The determination may be made by using an image. A technology obtained by applying Bluetooth, Wi-Fi (registered trademark), or a telephone line may be used as a substitute although the communication is wireless.

FIG. 5 is an explanatory diagram illustrating a state in which the treatment instrument TT is placed on a cart CA. For example, the cart CA may have a management unit configured to hold information related to the treatment instrument TT arranged on the cart CA, and the management unit may have a function of communicating with a treatment instrument communication unit (communication apparatus) 36 which will be described below, and transmitting the information related to the treatment instrument TT on the cart CA to the treatment instrument communication unit 36.

The display control apparatus for endoscopy 30 includes a control unit 31, an image obtaining unit 32, an image processing unit 33, a treatment candidate target detection unit 34, a treatment instrument decision unit 35, the treatment instrument communication unit 36, and a display control unit 37. Each of components of the control unit 31 and the display control apparatus for endoscopy 30 may be configured by a processor (one or more processors) using a CPU (central processing unit), an FPGA (field programmable gate array), or the like. The component may operate according to a program stored in a memory which is not illustrated to control each unit, or may achieve part or whole functions by an electronic circuit of hardware.

During the observation, the endoscope can use illumination light (e.g., daylight, white light), and narrow-band light or specific light (e.g., different wavelength distribution from the white light, monochromatic light). By changing the type of the light, the endoscope can obtain images. The processor can obtain information related to a surface of the treatment candidate target, a depth of the treatment candidate target, edge of the treatment candidate target. Based on the information, the processor can determine a whether treatment for the treatment candidate target is required, the scale of the treatment, and the type of the treatment (debridement, removal, local injection, biopsy, cauterization, hemostasis, marking, etc.) by changing the type of the light. Based on the determination, the processor can generate the guide information including recommendation of the treatment instrument.

The control unit 31 is configured to control an entirety of the display control apparatus for endoscopy 30 in an overall manner. The image obtaining unit 32 is configured to obtain the image information from the endoscope 20 upon endoscopy. The image processing unit 33 is configured to apply predetermined image signal processing on the image information obtained by the image obtaining unit 32. For example, the image obtaining unit 32 performs predetermined signal processing such as color adjustment processing, matrix conversion processing, noise removal processing, and various types of other signal processing on the picked-up image obtained by the image pickup device 22. The display control unit 37 is configured to supply an image (endoscopic image) obtained by the image processing by the image processing unit 33 to the display unit 41 which configures an output unit 40, and to cause the display unit 41 to display the image. The display unit 41 is, for example, display equipment having a display screen such as an LCD (liquid crystal display device). The one or more processors can be configured to process one or more color adjustment processing, matrix conversion processing, noise removal processing to the image.

Note that for ease of the explanation, a guide display technology of display control for endoscopy that is a feature of the present application is described in such a manner that the display unit 41 configured to display the endoscopic image is assumed, a display unit other than the above described display unit may be used. For example, a sub monitor installed to be adjacent to the display unit or wearable smart glasses may be used. It goes without saying that such an application is also assumed that a voice corresponding to the displayed guide information is outputted by a speaker or the like. Note that since a treatment candidate target of a specific image feature (feature) is detected based on a picked-up image obtained upon the endoscopy (which is equivalent to an image displayed on the display unit), in order to indicate to which part of the displayed image the display control corresponds, the guide display which is easily understood can be realized in some cases by using the same display unit by displaying a frame or indicating with use of a leader line or an arrow. For example, in a case where the image feature of the treatment candidate target is detected, and the guide display including information of a treatment instrument candidate (treatment instrument) suitable to be used for the above mentioned treatment is displayed on the display image, the display also provides accountability for a reason for issuing the guide.

The treatment candidate target detection unit 34 is configured to detect a treatment candidate target from the endoscopic image. For example, the treatment candidate target detection unit 34 may detect the treatment candidate target based on a specific image feature in the picked-up image obtained from the endoscope upon the endoscopy. For example, the treatment candidate target detection unit 34 may detect the lesion part included in the image to be set as the treatment candidate target by AI processing on the endoscopic image. The treatment candidate target detection unit 34 may detect the treatment candidate target included in the image by predetermined image analysis processing.

During the endoscopy, the endoscope images can be obtained at 60 frames per second (the number of frames depends on the endoscope). The endoscopy includes searching for the treatment candidate target, screening for the treatment candidate target, treating the treatment candidate target, adjusting the position of the distal end portion of the endoscope, and other operations. The searching, the screening, and the treating mainly performed while the endoscope is withdrawn. Therefore, huge number of images can be obtained in a single endoscopy. The processor can select the images including the treatment candidate target based on some features in the images, or some operation information of the endoscope among the huge number of images. To select the images including the treatment candidate target efficiently, the processor can use specific features obtained from the images or specific operation information of the endoscope. For example, when the user observes the treatment candidate target, the user is careful to find the feature or the treatment candidate target while keeping position around the particular part of the subject. As the result, some similar images can be obtained, the processor can determine that some similar images are likely to include the treatment candidate target.

Regarding the determination of similar images, for example, the processor can determine the image processing with respect to two or more images consecutively obtained. The image processing includes image matching with respect to the two or more images, and determine common feature among the two or more images. The common feature can be related to the treatment candidate target, or the same tissue around the treatment candidate target, the same angle of view. The determining that the common feature among the two or more images includes one or more of determination that number of pixels corresponding the common feature, determination that area of pixels corresponding the common feature, determination that ratio of pixels corresponding the common feature with respect to the whole pixels of the image. The processor can conduct the determination of the treatment candidate target when above determinations related to the image matching as trigger of the start of the determination of the treatment candidate target. The image feature can include information related to pixels corresponding a common feature between the first image and the second image, the common feature is obtained from image matching.

Alternatively, instead of the image processing of the two or more images consecutively obtained, image matching can be performed on the image obtained by the endoscopy and the template image (sample image previously obtained) of the treatment candidate target to determine image feature that is common to two or more images. The image feature can include information related to pixels corresponding a common feature between the first image and the second image, the common feature is obtained from image matching.

Furthermore, the other than the images, the processor can use an information related to the fluid supply, or any kind of pre-treatment as the trigger of the start of the determination of the treatment candidate target. The information related to the fluid supply can be obtained from the signal of the switch or the images. The placement of the distal end of the endoscope is fixed, so the treatment instrument can be determined from the area in the image. Once the treatment instrument is determined, it is judged that the procedure has started, and the position where the tool acts can be determined as a treatment candidate target.

Furthermore, the trigger of the start of the determination of the treatment candidate target can be a blurring amount of the distal end portion of the endoscope. the blurring amount is obtained by the sensor on the distal end portion of the endoscope or the image matching of the two or more images obtained by endoscopy. Alternatively, since the user operates the supply/suction of the air/water by button or switch, the time and amount are calculated based on the operation signals of the button or switch. The calculated time and amount can then be compared to a predetermined value and used as one of the factors for judging treatment candidate target. The calculated time or amount is smaller or larger than the predetermined value can be the trigger. The image feature includes a blurring amount of a distal end of the endoscope.

The treatment instrument decision unit 35 is configured to decide the treatment instrument candidate corresponding to the treatment candidate target detected by the treatment candidate target detection unit 34. The treatment instrument candidate decided by the treatment instrument decision unit 35 enables an appropriate treatment for the treatment candidate target. The treatment instrument decision unit 35 decides the treatment instrument candidate based on the image feature of the treatment candidate target. For example, the treatment instrument decision unit 35 may decide the treatment instrument candidate based on the image feature of the treatment candidate target such as a shape, a size, or a color. For example, in a case where the treatment candidate target is a polyp, according to a nature of the polyp, that is, a shape, a size, a color, or the like of the polyp, a type or a size of the snare that is the treatment instrument may be determined. The feature indicates one or more of a location of the treatment target, a shape of the treatment target, a size of the treatment target, a color of the treatment target, an edge of the treatment target, a focused area of the treatment target, concavity and convexity about the treatment target, a blood pattern about the treatment target, a blood arrangement about the treatment target or a distance between the endoscope and the treatment target. The feature can be obtained by an image pick up device.

For example, in a case where it is supposed that an optical property of the image pickup device 22 is substantially constant, when mutual endoscopic images are in focus, it is conceivable that the mutual endoscopic images are obtained through image pickup from approximately a same specific distance. The endoscope 20 capable of measuring a distance also exists. Therefore, the treatment instrument decision unit 35 can determine the shape and the size of the actual treatment candidate target by the treatment candidate target in the endoscopic image.

After the determining of one or more of the treatment candidate target, the pre-treatment, the protruding the treatment instrument, or the blurring amount, the treatment instrument can be determined as the candidate. The data indicating relationship between the treatment instrument and the feature of the treatment target is obtained from the endoscopic images of cases in which the procedure has been completed. The data of the relationship can be used for creation for database and inference model. The relationship can be inferred by the inference model. Because depend on the case, the treatment instrument can be differed even though the treatment targets can be categorized same group on the pathological diagnosis.

Further, in addition to the relationship, the data can include one or more of the model number and specifications. The data can be used for creation for database and inference model.

Further details will be explained. In the endoscopic images obtained by time-series, there are images including the treatment target from one second to several seconds. This behavior is caused by the user to recognize the treatment target. After that, there are other images including the treatment instrument. In another images, the treatment instrument move toward the treatment target from the specific position. This behavior is because of the treatment instrument is inserted into the subject through the fixed opening of the endoscope.

The processor can determine the object that move toward the treatment target as the treatment instrument, and determine the type of the treatment instrument based on the size, color, shape of the object. In addition to or instead of the type of the treatment instrument, the processor can determine the model number and/or the specifications.

Therefore, this application discloses the processor configured to detect the first region of the first treatment target indicating the image feature from the series of first endoscopic images obtained during the endoscopy; based on the image feature, determine the first image including the second region of the second treatment target from the series of second endoscopic images being obtained prior to the series of first endoscopic images; determine the second image subsequent to the first image, and the second image including the region of the treatment instrument; determine the treatment instrument candidate based on the treatment instrument in the second image; and display the guide information related to the treatment instrument candidate.

Further this application discloses the endoscopic control method, comprising detecting a treatment target of a first image feature from first images obtained from an image pick-up device of an endoscope during a first endoscopy; determine a second image feature based on the first image feature, the second image feature is obtained from a second images during a second endoscopy being conducted prior to the first endoscopy; determining a treatment instrument based on the second image feature, the treatment instrument being included in third image subsequent to the second image; and determining a treatment instrument candidate corresponding to the treatment instrument; displaying guide information including information related to the treatment instrument candidate. The method can comprise referencing a database storing the second image feature associated with the treatment instrument.

An inference model (treatment instrument inference model) used in AI processing for the treatment instrument decision unit 35 to decide a treatment instrument candidate corresponding to the treatment candidate target may be created by using training data based on the endoscopic image obtained in the endoscopy. For example, the treatment instrument decision unit 35 may decide the treatment instrument candidate by using the inference model (treatment instrument inference model) obtained by learning the training data obtained by annotating the treatment instrument appearing in the endoscopic image with respect to a series of frames of the endoscopic image obtained by the endoscopy. For example, in a case where a treatment such as resect is performed by using a treatment instrument on a lesion part or the like that is the treatment candidate target upon endoscopy, images of the treatment instrument are picked up only during a period in which the resection of the lesion part is performed and periods before and after the period. Therefore, by detecting the treatment instrument appearing in the endoscopic image during the above mentioned periods and applying annotation, the training data can be created.

The treatment instrument decision unit 35 supplies information indicating that the decided treatment instrument candidate is a treatment instrument suitable to the treatment on the treatment candidate target to the display control unit 37. The display control unit 37 supplies, to the display unit 41, display data of the guide display for recommending the treatment instrument candidate to be used in the treatment on the treatment candidate target detected in the treatment candidate target detection unit 34. Thus, the guide display indicating the treatment instrument candidate suitable to the treatment is displayed on the display screen of the display unit 41.

Furthermore, in the present embodiment, not only the guide display for recommending the use of the treatment instrument candidate decided by the treatment instrument decision unit 35 may be simply displayed, but also information on how to use the treatment instrument candidate, a degree of difficulty of the treatment, or the like may be displayed as the guide display. For example, a treatment time period spent for the treatment of the lesion part may be obtained based on the endoscopic image obtained by the endoscopy, and information on a degree of difficulty analogized based on the treatment time period at the time of display of the treatment instrument candidate may be displayed. The one or more processors can be configured to calculate a treatment time period for treating a lesion part based on an endoscopic image obtained from the endoscope during the endoscopy, and determine a degree of difficulty based on the treatment time period and the treatment instrument. The guide information includes the degree of difficulty.

In the present embodiment, the treatment instrument decision unit 35 may be configured to display the guide display including information of a location where the treatment instrument candidate exists. For example, the treatment instrument decision unit 35 may determine whether or not the treatment instrument candidate exists in an inspection room or the like. In a case where the treatment instrument candidate exists, the guide display may be displayed to recommend the use of the treatment instrument candidate and to indicate a location where the treatment instrument candidate exists, a time period required to prepare the treatment instrument candidate, or the like.

For example, by communicating with the treatment instrument TT, the treatment instrument communication unit 36 may obtain the information of the location where the treatment instrument candidate decided by the treatment instrument decision unit 35 exists. Accordingly, the treatment instrument decision unit 35 determines whether or not the treatment instrument candidate is placed, for example, on the cart CA. For example, by directly communicating with the RFID tag TTt provided in the treatment instrument TT, the treatment instrument communication unit 36 may be able to obtain information related to the treatment instrument existing on the cart. The treatment instrument communication unit 36 may communication with a treatment instrument other than the treatment instrument in the close location by an appropriate communication protocol. The treatment instrument communication unit 36 may be able to communicate with an apparatus configured to manage a treatment instrument and obtain information related to treatment instruments stored inside a hospital other than the inspection room or treatment instruments existing outside the hospital, for example. The endoscopic control apparatus 30 can comprise a communication apparatus 36 configured to wirelessly communicate with an electronic tag (TTt) provided in the treatment instrument. The one or more processors can be configured to check for a presence of the treatment instrument by communication between the communication apparatus and the electronic tag.

The treatment instrument communication unit 36 supplies the obtained information related to the treatment instrument candidate to the treatment instrument decision unit 35. In a case where the treatment instrument candidate exists on the cart in the inspection room, for example, the treatment instrument decision unit 35 supplies, to the display control unit 37, information indicating that the treatment instrument candidate is suited to the treatment on the treatment candidate target. The display control unit 37 supplies, to the display unit 41, the display data of the guide display for recommending the use of the treatment instrument candidate for the treatment on the detected treatment candidate target. Thus, the guide display indicating the treatment instrument candidate which exists on the cart and which is suitable to the treatment is displayed on the display screen of the display unit 41.

In a case where the treatment instrument candidate does not exist on the cart in the inspection room, for example, but exists in a storage location inside the hospital, the treatment instrument decision unit 35 supplies, to the display control unit 37, the information indicating the treatment instrument candidate suitable to the treatment on the treatment candidate target, and information with regard to the storage location, the time period spent to prepare the treatment instrument candidate, and the like. The display control unit 37 supplies, to the display unit 41, the display data of the guide display recommending the use of the treatment instrument candidate for the treatment on the detected treatment candidate target, and indicating the storage location and the time period for the preparation of the treatment instrument candidate, and the like. Thus, the guide display indicating the treatment instrument candidate suitable to the treatment and the storage location is displayed on the display screen of the display unit 41.

In a case where it is determined that a plurality of treatment instrument candidates are suited to the treatment based on the endoscopic image, the treatment instrument decision unit 35 may be configured to select a treatment instrument in a closest location, for example, the treatment instrument existing on the cart, as the treatment instrument candidate, and display the guide display related to the treatment instrument candidate.

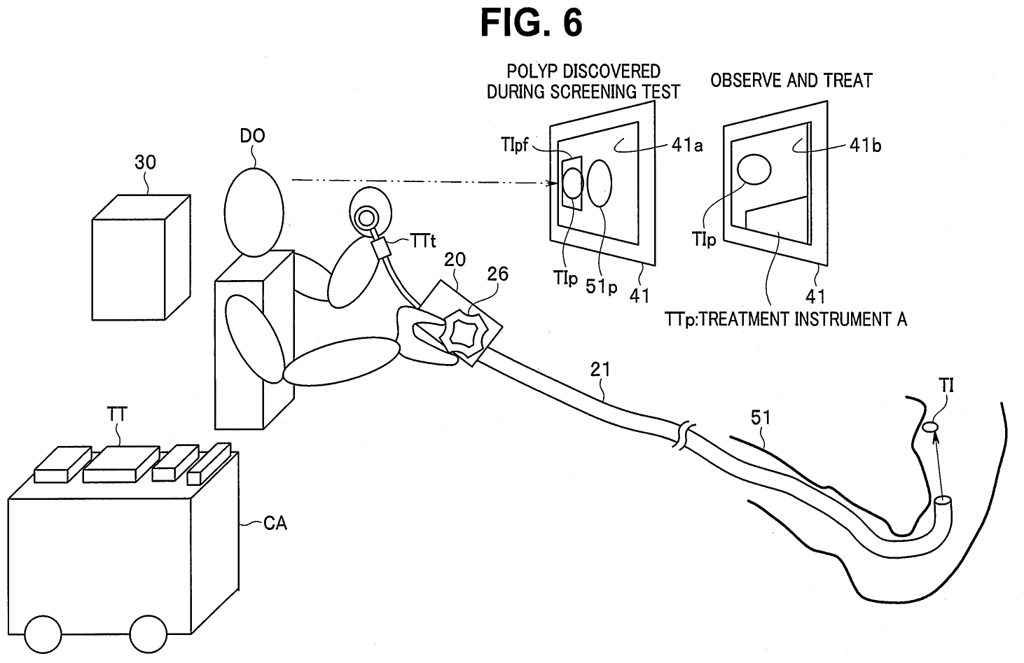

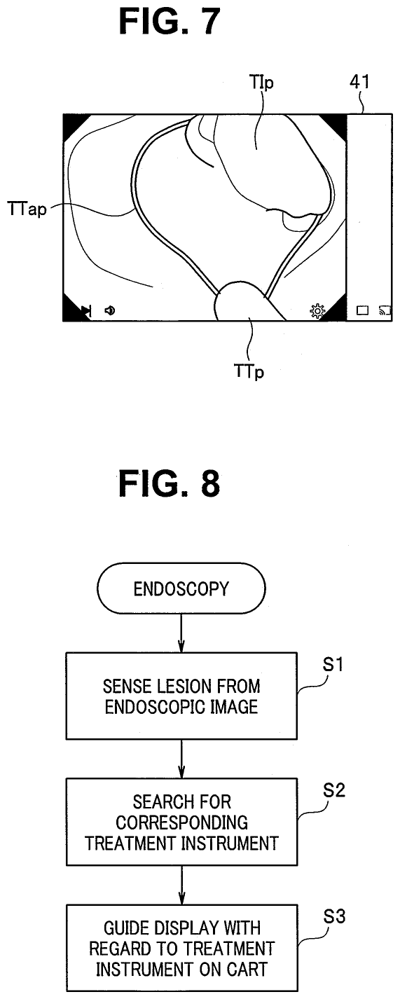

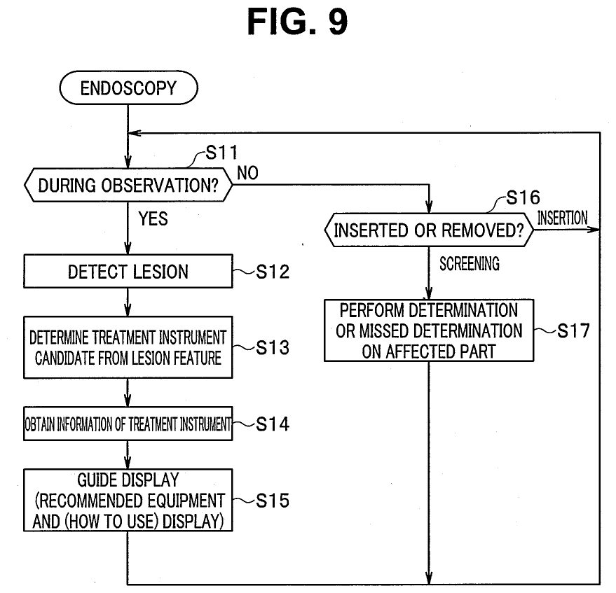

Next, an operation of the thus configured embodiment will be described with reference to FIG. 6 to FIG. 9. FIG. 6 is an explanatory diagram illustrating a situation of the endoscopy. FIG. 7 is an explanatory diagram for describing a treatment on the treatment candidate target. FIG. 8 is a flowchart for describing an outline of the treatment in the endoscopy, and FIG. 9 is a flowchart for describing operation in the endoscopy.

In the endoscopy (for example, a screening test) in the present embodiment, the flow of FIG. 8 is adopted. In other words, a treatment candidate target such as a polyp is discovered from the endoscopic image during the screening test (S1). A doctor DO selects a treatment instrument for treating the discovered treatment candidate target. In this case, the display control apparatus for endoscopy 30 searches for the treatment instrument candidate suited to the treatment candidate target (S2), and presents the treatment instrument candidate on the cart which is obtained through the search, for example, to the doctor DO as the guide display (S3). The display control apparatus for endoscopy 30 also displays the guide display as to whether or not the treatment instrument candidate exists on a nearby cart. The doctor DO selects the treatment instrument for treating the treatment candidate target by referring to the guide display. Note that in a case where the treatment instrument candidate suited to the treatment candidate target is not on the nearby cart, a time period spent to transport the treatment instrument candidate from the outside of the inspection room is displayed to facilitate the doctor to decide the treatment instrument on the treatment candidate target and make a decision with regard to implementation of the treatment. The endoscopic control apparatus 30 comprises one or more processors configured to process image matching with respect to a first image and a second image to determine an image in the first image and the second image, the first image obtained from an endoscope during an endoscopy, the image feature comprises information related to common pixels among the first image and the second image, detect a treatment target based on the feature obtained from the image matching (S1), determine a treatment instrument based on the detected treatment target (S2), and generate guide information (S3). The guide information includes information related to the treatment instrument. The one or more processors is configured to determine whether the treatment instrument is present on a treatment instrument cart CA. The one or more processors can be configured to in response to the determining that the treatment instrument is present on the treatment instrument cart CA, output the guide information including the treatment instrument presented on the treatment instrument cart CA. The one or more processors can be configured to in response to the determining that the treatment instrument is not present on the treatment instrument cart CA, determine whether a substitute treatment instrument is present on the treatment instrument cart CA or not, the guide information includes information related to the substitute treatment instrument. The one or more processors can be configured to display the generated guide information. An endoscope system can comprise the endoscopic control apparatus 30, and the endoscope 20. The endoscope system can comprise display equipment 40. The medical system can comprise the endoscope system display equipment 40, and the endoscope 20.

In the example of FIG. 6, in an endoscopy room, a patient is lying on a bed (not illustrated), and the doctor DO inserts the insertion portion 21 of the endoscope 20 to a lumen 51 of the patient to implement the endoscopy (for example, the screening test). The doctor DO causes the insertion portion 21 to be bent by operating a bending knob 26 and causes the insertion portion 21 to advance or retreat to insert the distal end of the insertion portion 21 to the lumen 51. An image of the lumen 51 is picked up by the image pickup device 22 provided at the distal end of the insertion portion 21. The image (endoscopic image) obtained by the image pickup by the endoscope 20 is supplied via a cable (not illustrated) to the display control apparatus for endoscopy 30. The endoscopic image processed by the display control apparatus for endoscopy 30 is displayed on the display screen of the display unit 41.

The display unit 41 is arranged within a field of view of the doctor DO. As indicated by a double-dashed arrow representing a line of sight, the doctor DO checks the endoscopic image displayed on a display screen 41a of the display unit 41, and performs the endoscopy. The cart CA is arranged in a vicinity of the doctor DO, and a plurality of treatment instruments TT are placed on the cart CA.

FIG. 6 illustrates two display screens 41a and 41b. The display screens 41a and 41b do not represent different arrangement locations of the display unit 41, but represent screens at different timings on the display unit 41. The display screen 41a is an example at a time when tissue TI such as a polyp is discovered during the screening test, and an image of the tissue TI of the patient is picked up by the endoscope 20. An image 51p on a deep side of the lumen 51 and an image TIp of the tissue TI are displayed on the display screen 41a.

The doctor DO recognizes, from the image TIp displayed on the display screen 41a, the tissue TI such as the polyp exists inside the lumen of the patient, and decides a treatment on the tissue TI. In this case, it is possible to refer to the guide display by the display control apparatus for endoscopy 30.

In step S11 of FIG. 9, the control unit 31 of the display control apparatus for endoscopy 30 determines whether or not an observation by the endoscope 20 is being performed. In the case of the observation being performed (YES in S11), the control unit 31 detects a treatment candidate target (treatment target) such as a lesion part (S12). In other words, the control unit 31 controls the treatment candidate target detection unit 34 such that the lesion part such as the polyp is detected from the endoscopic image as the treatment candidate target. The one or more processors can be configured to determine whether an observation is performed during the endoscopy (S11). The treatment target is detected during the observation.

The detection is allowed depending on whether or not a specific image feature exists when an image of the treatment candidate target is picked up. For example, when a flat part is swollen, a shadow can be created when the swollen part is illuminated. The image feature changes in a treatment target site. In addition to the above mentioned phenomenon, a color may change due to swelling or a poor blood circulation. The determination can also be performed by an arrangement, a pattern, or the like of blood vessels in detail. A large number of training data obtained by specifying (annotating) by surrounding such a treatment target portion with a frame may be prepared and trained to create an inference model. The thus obtained inference model is caused to detect the treatment candidate target. At this time, the inference model configured to identify lesions into a plurality of types can also be created by annotating what kind of lesion the treatment candidate target is (lesion classification may be written as texts, or may be classified by flags, symbols, or the like). The one or more processors can be configured to determine the treatment instrument using an inference model. The inference model is obtained from learning training data obtained by annotating a treatment instrument appearing in an endoscopic image obtained from the endoscope during the endoscopy. Alternatively, the one or more processors can be configured to determine the treatment instrument based on the detected treatment target and a database reference.

When a lesion is detected, the display control unit 37 displays a frame display TIpf indicating a location of a treatment candidate target that is a lesion part based on a detection result of the treatment candidate target detection unit 34. The treatment instrument decision unit 35 determines the treatment instrument candidate suitable to the treatment of the treatment candidate target by AI processing using the treatment instrument inference model, for example, based on a feature of the lesion, that is, a feature of the endoscopic image (S13).

The determination method does not need to be limited to the AI processing using the treatment instrument inference model. Such a database is provided in parallel that lesions can be identified into a plurality of types, and it is possible to search for an appropriate treatment instrument for each of identification results. A result of the search may be set as the judgement result. The database may be created in such a manner that information is so organized that at the time of the search for a specific treatment instrument, treatment instruments with similar functions and performances and the like can also be searched for. At the time of the search for the treatment instrument, information related to the treatment instrument may be searched for in still another database.

The treatment instrument decision unit 35 outputs, to the display control unit 37, information for presenting the determined treatment instrument candidate to the doctor DO. Accordingly, the display control unit 37 displays the guide display indicating the treatment instrument candidate on the display unit 41. The display screen 41b illustrates a display example in this case. In the example of the display screen 41b in FIG. 6, as the treatment instrument candidate, a “treatment instrument A” is displayed in a form of the guide display. Note that the treatment instrument decision unit 35 may cause the guide display to be displayed using a search result on whether or not the treatment instrument candidate exists on the cart CA.

In other words, in this case, to obtain information of the treatment instrument candidate, for example, the treatment instrument communication unit 36 communicates with the treatment instrument candidate (S14). Through the communication, for example, the treatment instrument communication unit 36 recognizes that the treatment instrument candidate exists on the cart CA. Alternatively, the treatment instrument communication unit 36 recognizes that the treatment instrument candidate does not exist on the cart CA. Note that in a case where the treatment instrument candidate exists inside the hospital but outside the inspection room, the treatment instrument communication unit 36 can also obtain information on the location where the treatment instrument candidate exists and the time period needed to prepare the treatment instrument candidate.

The treatment instrument decision unit 35 supplies, to the display control unit 37, information for causing the guide display indicating the treatment instrument candidate placed on the cart CA to be displayed, for example, based on the information obtained by the treatment instrument communication unit 36. Accordingly, the display control unit 37 displays the guide display indicating recommended equipment for the treatment instrument candidate on the display screen 41b of the display unit 41 (S15). In this case, the display control unit 37 may perform the guide display with regard to how to use the treatment instrument candidate.

The doctor DO refers to the guide display to select the treatment instrument. For example, the doctor DO selects the treatment instrument TT which is on the cart CA and which has the electronic tag TTt, inserts the selected treatment instrument TT from the insertion slot of the endoscope 20, and causes the treatment instrument to protrude from the opening portion 25 of the distal end surface of the insertion portion 21 as indicated by an arrow of FIG. 6 to perform the treatment on the tissue TI.

FIG. 7 illustrates a display example on the display unit 41 at the time of a treatment. In the example of FIG. 7, by an image TTp of the treatment instrument TT, an image TTap depicting a wire of the distal end portion, and the image TIp of the tissue TI, it is found that the doctor DO hooks the wire of the distal end portion of the treatment instrument TT on the tissue TI such as the polyp. The doctor DO performs resect or the like on the tissue TI such as the polyp by using the treatment instrument TT.

In a case where it is determined in S11 that the observation is not being performed, the control unit 31 determines whether the treatment instrument is in the process of being inserted or removed in S16. The control unit 31 returns the processing to S11 when the treatment instrument is in the process of being inserted. When the treatment instrument is in the process of being removed, that is, at the time of the screening, the control unit 31 performs determination or missed determination on the affected part in the next step S17.

Note that in the above description, by way of the guide display, the description has been provided where the guide display for presenting the presence or the location of the treatment instrument candidate or the time period for the preparation or the guide display on how to use the treatment instrument candidate is displayed. Furthermore, a guide display on a precaution or the like after the use of the treatment instrument candidate may be performed. For example, not only information on how to use but also information on a precaution on how to handle with regard to each of treatment instruments such as how to dispose after the use may be obtained by the treatment instrument communication unit 36. Accordingly, the treatment instrument decision unit 35 can include the above mentioned information in the guide display.

In this manner, according to the present embodiment, in a case where the doctor selects the treatment instrument, the display control apparatus for endoscopy displays the guide display for presenting the treatment instrument candidate suited to the detected treatment candidate target to the doctor. By referring to the guide display, the doctor can easily recognize the treatment instrument candidate suited to the detected treatment candidate target. In a case where the treatment instrument candidate suited to the treatment candidate target exists on a nearby cart, the display control apparatus for endoscopy can display the information of the treatment instrument candidate as the guide display. Furthermore, in a case where the treatment instrument candidate suited to the treatment candidate target does not exist on the nearby cart, the display control apparatus for endoscopy can present a location where the treatment instrument candidate exists and a time period for the preparation, and the determination such as the selection of the treatment instrument in a case where the doctor performs the treatment can be effectively assisted.

### (Treatment Instrument Inference Model)

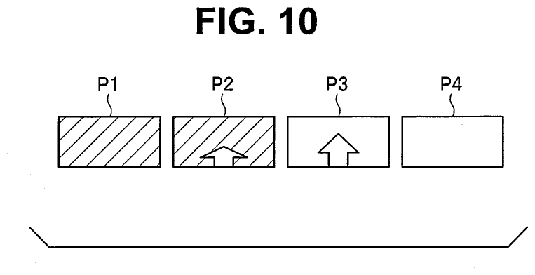

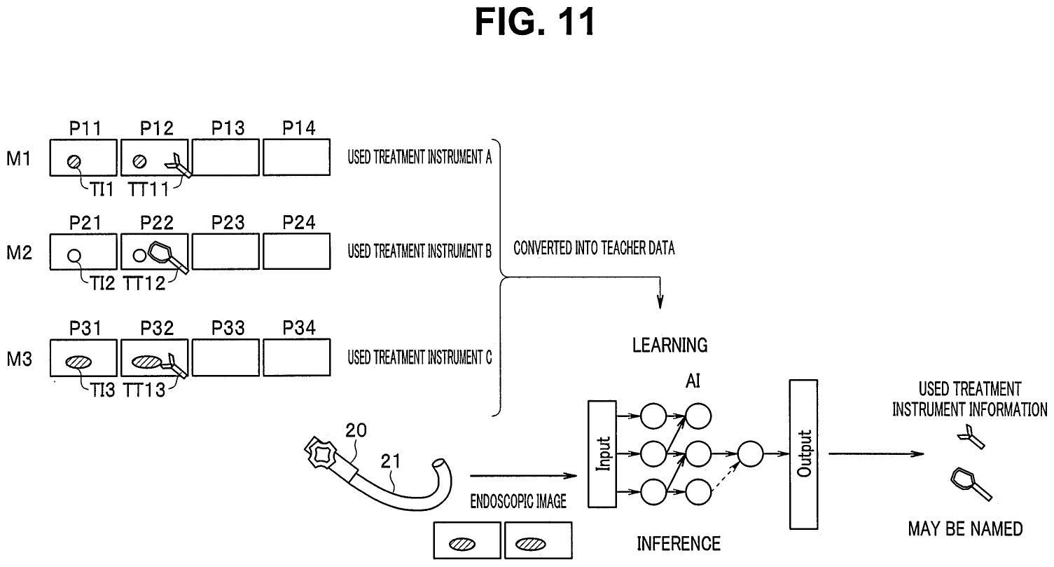

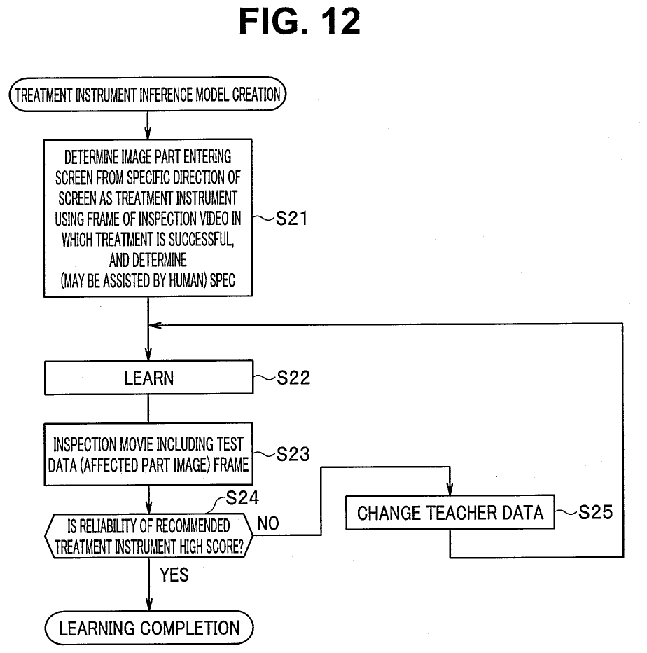

FIG. 10 and FIG. 11 are explanatory diagrams for describing a generation method of a treatment instrument inference model. FIG. 12 is a flowchart illustrating a creation (learning) method of the treatment instrument inference model. An endoscopic control method comprises processing image matching with respect to a first image and a second image to determine an image feature in the first image and the second image, the first image obtained from an endoscope during an endoscopy, the image feature comprises information related to common pixels among the first image and the second image, detecting a treatment target of the image feature obtained from the image matching, determining a treatment instrument based on the image feature of the treatment target, displaying guide information. The guide information includes information related to the treatment instrument. The determining that the treatment instrument can include detecting frames from among continuous frames from a plurality of images obtained by the endoscope during endoscopy and in which a treatment instrument appears, and determining the treatment instrument using an inference model applied to the detected frames. The inference model is obtained from learning training data obtained by annotating the treatment instrument appearing in a series of frames of an endoscopic image obtained from the endoscope during the endoscopy. The inference model correlates a relationship between a treated treatment candidate image and the treatment instrument used.

FIG. 10 illustrates a series of frames P1, P2, and so on of an endoscope movie. An arrow in the frames P2 and P3 represents that the treatment instrument enters an image. As illustrated in FIG. 3, for each type or model of the endoscope, a positional relationship between the observation window 23 and the opening portion 25 on the distal end surface of the insertion portion 21 is fixed. In a state in which an image of a target object is picked up via a distal end observation window for image pickup, in a case where the treatment instrument protrudes from the distal end opening portion 25 towards the target object, which position (direction) in the endoscopic image in which the treatment instrument appears is determined. Therefore, it is relatively easy to determine, from changes in the images of the series of frames P1, P2, and so on, the treatment instrument which enters the endoscopic image.

Namely, an object appearing in the image is regarded as the treatment instrument by following such a logic. Furthermore, other conditions may be added to the logic. Namely, the treatment instrument appears to treat the target object being observed by the image pickup unit. Therefore, the following conditions are conceivable that an (image) position of the treatment target object in the image is not changed, the image feature of the target object has a feature worth to be treated, the observation is performed at a short distance to cause the treatment instrument to approach the target object, and the like. By using the feature of the image change at a time when the treatment instrument appears in the image based on such a logic, the training data for inference model corresponding to the treatment instrument can be created relatively easily. Images of the endoscope are continuous still images or a video. When shooting and recording are performed from the start to the end of the inspection, a large number of image frames are produced. It is difficult to pick out appropriate image frames for training data from the large number of image frames, but with such a way of thinking, necessary images can easily be selected. Namely, such a logic can be used that the treatment instrument (image) is determined based on the feature of the image appearing in a specific direction in the image frame, and the type of the treatment instrument is determined.

By using such continuous image frames (plurality of image frames), there is an advantage that it is possible to determine which part is a background in the image and which part is an image part of the treatment instrument from a motion of the treatment instrument or the like. An image of a feature of an infiltration into a specific background (such as a wall of a digestive tract with an affected part) corresponds to the treatment instrument. A part changing over time by the treatment by the treatment instrument is a part that is to be treated, and it is possible to perform processing by assuming that the feature of the image of the part is the treatment target portion.

Namely, with such devices, an advantage is attained that the infiltration of the treatment instrument can be determined, the image feature of the treatment instrument can be accurately determined, and the image feature of the target that is to be treated can be accurately found by the direction in which the treatment instrument approaches, the image change before and after the treatment, or the like. When the image feature corresponding to the target that is to be treated is found, the treatment instrument used at the time can be estimated or inferred reversely. Note that herein, a shape, a color, a size, shading, a color change, a specific pattern, glossiness, and the like are assumed as the image feature.

The training data for generating the treatment instrument inference model is created by using a movie at the time of the actual endoscopy as a base. An endoscope movie M1 of FIG. 11 indicates that in a series of frames P11 to P14, tissue TI1 such as a polyp is treated by a treatment instrument TT11 and disappears. Similarly, an endoscope movie M2 indicates that in a series of frames P21 to P24, tissue TI2 such as a polyp is treated by a treatment instrument TT12 and disappears. An endoscope movie M3 indicates that in a series of frames P31 to P34, tissue TI3 such as a polyp is treated by a treatment instrument TT13 and disappears.

In step S21 of FIG. 12, by using an inspection video (endoscope movie) in a case where the treatment is successful, from a comparison between images before and after the polyp or the like disappears, an image part entering from a specific location of the image before the polyp or the like disappears is determined as the image of the treatment instrument. With regard to each of the endoscope movies M1, M2, and so on, annotation for assigning a treatment instrument name or the like for discriminating the treatment instrument is implemented to be converted into training data. In this case, for each treatment instrument, not only the treatment instrument name but also a specification or the like may be annotated.

In this manner, when an inference model corresponding to the treatment instrument is created, a timing at which the treatment on the specific target object is performed may be determined by a plurality of image frames obtained from the image pickup device of the endoscope apparatus. An image frame serving as the training data may be selected based on the feature of the image appearing in the specific direction in the image frame at timings before and after the timing at which the treatment on the specific target object is performed. An object appearing in the image may be set as a treatment instrument, and a type of the treatment instrument may be determined. Information of the treatment instrument may be annotated to the image frame. Deep learning is performed with the thus obtained annotated training data to generate the inference model.

In this manner, the training data for generating the treatment instrument inference model is obtained in such a manner that the control unit 31 configuring a training data creation apparatus determines a timing at which the treatment on the specific target object is performed by a plurality of image frames obtained from the image pickup device of the endoscope apparatus, determines a type of the treatment instrument based on the feature of the image appearing in the specific direction in the image frame at timings before and after the timing at which the treatment on the specific target object is performed, and annotates information of the treatment instrument to the image frame. Note that the annotation may be implemented by human judgement, or may be implemented by the AI processing by using the inference model obtained by learning the training data generated in advance based on the image of the treatment instrument and the annotation result.

With respect to the images obtained by the endoscope, the processor is configured to
(1) determine the treatment instrument protruded from the opening of the distal end portion of the endoscope, this determination can be performed by the image processing;
(2) determine the treatment candidate target;
(3) determine the treatment time period that the treatment candidate target is treated by the treatment instrument;
(4) annotate the treatment instrument as the feature of the image;
(5) generate an inference model with the image as input and the instrument type and treatment time period as output by using the type of the treatment instrument, the treatment time period, and annotated image as training data.

Alternatively, with respect to the images obtained by the endoscope, the processor is configured to
(1) determine the treatment instrument protruded from the opening of the distal end portion of the endoscope, this determination can be performed by the image processing, this determination can reference the database;
(2) determine the treatment candidate target;
(3) annotate the treatment instrument as the feature of the image;
(4) generate an inference model with the image as input and the instrument type as output by using the type of the treatment instrument, and annotated image as training data.

Furthermore, the image, the treatment instrument, the treatment time period that are obtained can be used for generating the database. The database can be used for determination of the degree of the difficulty of the using of the treatment instrument.

In this manner, the present specification also includes such an aspect that the good training data can easily be selected, and the excellent inference model can quickly be created. Namely, according to the technology of the display control for endoscopy, a treatment candidate target of a specific image feature is detected based on a picked-up image obtained upon endoscopy. To infer a corresponding treatment instrument candidate based on the image feature of the treatment candidate target, from among continuous frames configuring a plurality of movies upon the endoscopy which are obtained in advance, frames in which the treatment instrument appears which appear in the respective endoscopic images are detected. An inference is performed to determine the treatment instrument candidate by using an inference model obtained by learning a relationship between a treated treatment candidate target image and the treatment instrument used upon the treatment by using a training data group obtained by annotating an image part corresponding to the treatment instrument from the frames in which the treatment instrument appears. A guide display including information of the treatment instrument candidate inferred when the endoscopic image is inputted is displayed. Namely, the treatment instrument candidate is determined by using a result of the inference in which an image frame (image frame including an image of the treatment candidate target of the specific image feature) obtained upon endoscopy which is about to be performed is inputted to the inference model obtained by learning such that the relationship between the treated treatment candidate target image and the treatment instrument used upon the treatment becomes an input and output relationship.

A large amount of thus generated training data is supplied to an inference engine which is schematically illustrated by an input layer (input), an output layer (output), and neurons to perform learning (S22), and a treatment instrument inference model is generated. When the learning is finished to some extent, a test is implemented. In other words, in S23, an inspection video including a frame of an affected part image is inputted as test data. The test data is an unknown image. In S24, the test data is applied to a neural network (S12). As a result, information of the treatment instrument candidate that is a recommended treatment instrument is obtained as a recognition result from the neural network. In S24, it is determined on whether reliability of the treatment instrument candidate is a high score or not (whether the test is successful or not). In a case where the score of the reliability of the treatment instrument candidate is not a high score, the training data is changed (S25), and the learning is performed again (S22). In a case where the score of the reliability of the treatment instrument candidate is a high score, it is regarded that the learning is completed.

The treatment instrument decision unit 35 may adopt the thus generated treatment instrument inference model. In this case, the treatment instrument decision unit 35 stores a plurality of frames of the endoscope movie from the endoscope 20 in the memory which is not illustrated, and supplies the plurality of frames to the treatment instrument inference model. Accordingly, in a case where a polyp or the like is included in an image of each frame, information related to the treatment instrument candidate corresponding to the polyp or the like can be obtained.

### Second Embodiment

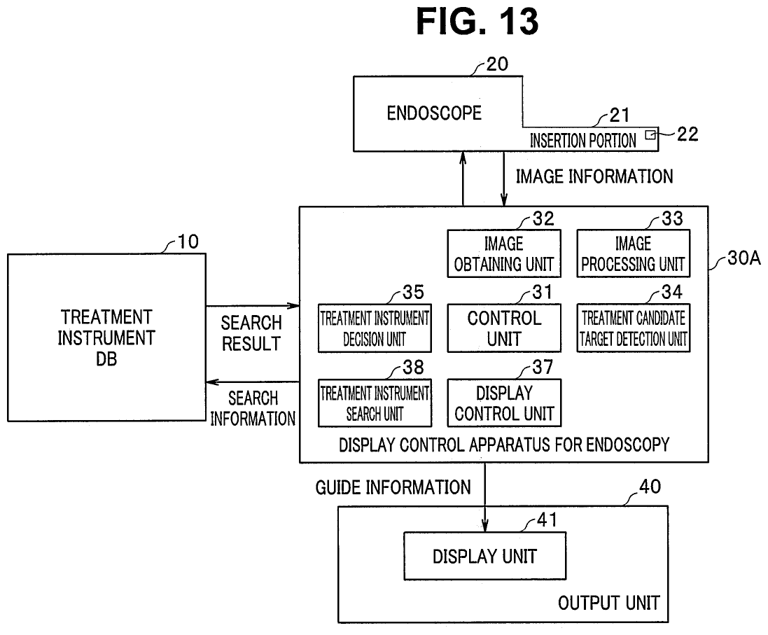

FIG. 13 is a block diagram illustrating the display control apparatus for endoscopy according to a second embodiment. In FIG. 13, a same component as the component in FIG. 1 is assigned with a same reference sign, and a description of the component is not repeated. The present embodiment is intended to enable presentation of a treatment instrument candidate suited to the treatment candidate target by referring to treatment instrument database (DB) configured to store information related to a treatment instrument.

A display control apparatus for endoscopy 30A in the present embodiment is different from the display control apparatus for endoscopy 30 of FIG. 1 in that a treatment instrument search unit 38 is adopted instead of the treatment instrument communication unit 36.

Note that treatment instrument DB 10 may be arranged in any location as long as the treatment instrument DB 10 is accessible from the display control apparatus for endoscopy 30A, and may be provided in a server on cloud, for example.

In the treatment instrument DB 10, information related to a treatment instrument usable in the endoscopy and treatment instruments usable in various types of treatments including a treatment performed upon the endoscopy is held. With regard to each treatment instrument, the treatment instrument DB 10 may be configured to store information through classification by distance from the inspection room to the treatment instrument, such as treatment instruments that can be ordered from manufacturers, treatment instruments stored in the hospital, or treatment instruments placed on the cart arranged in the inspection room.

For example, the treatment instrument DB 10 may include a communication apparatus which is not illustrated. With the communication apparatus, the treatment instrument DB 10 can access the database in the hospital and obtain information on the treatment instrument stored in the hospital. In a case where an electronic tag such as the RFID is attached to the treatment instrument, by communicating with the electronic tag of each treatment instrument, the treatment instrument DB 10 can also grasp the treatment instrument placed on the cart in the inspection room. The cart may have a function of holding the information related to the treatment instrument arranged on the cart and communicating with the treatment instrument DB 10 to transmit the information related to the treatment instrument on the cart to the treatment instrument DB 10.

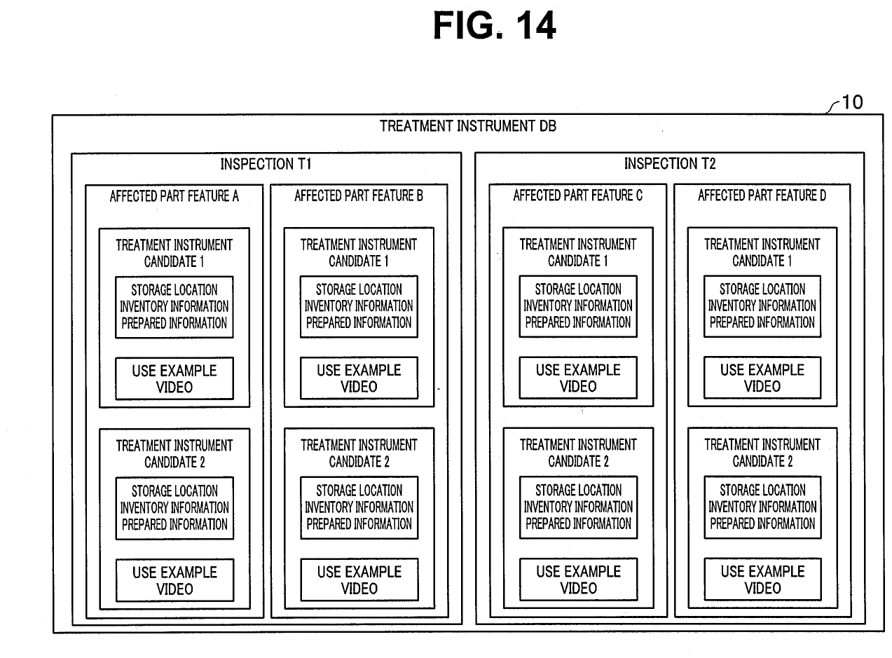

FIG. 14 is an explanatory diagram illustrating an example of information registered in the treatment instrument DB 10. FIG. 14 corresponds to two types of endoscopy T1 and endoscopy T2 and exemplifies content of the treatment instrument candidates registered in the treatment instrument DB 10. In the example of FIG. 14, the treatment instrument DB 10 stores information of the treatment instrument candidates corresponding to an affected part feature A and an affected part feature B with regard to the inspection T1, and stores information of the treatment instrument candidates corresponding to an affected part feature C and an affected part feature D with regard to the inspection T2. In the example of FIG. 14, an example is illustrated in which information of two treatment instrument candidates are stored with regard to each of the affected part features A to D. The information of each treatment instrument candidate includes storage location information, inventory information, and prepared information. The information may also include information on whether the treatment instrument candidate currently exists in the storage location or not (whether the treatment instrument candidate is taken out or not). Furthermore, the information of each treatment instrument candidate may include information of a use example, for example, video information, for each of the treatment instruments. When such information can be displayed as a reference, the information helps when the treatment instrument is used, and a smooth treatment can be carried out. The inspection does not take time, and the patient does not need to be restrained for a long period of time.

By storing information on treatment candidate target, treatment instrument and medical case in moves or images in the database as examples during the endoscopy, it is possible to search for corresponding treatment instrument for treatment candidate target and medical case in image obtained during another endoscopy.

The treatment instrument search unit 38 accesses the treatment instrument DB 10, and supplies information with regard to the treatment instrument candidate decided by the treatment instrument decision unit 35 to the treatment instrument DB 10 as search information to obtain a search result from the treatment instrument DB 10. Information of the search result includes information indicating whether the treatment instrument candidate exists in any location, for example, information indicating whether or not the treatment instrument candidate exists on the cart which is not illustrated and which is arranged in the inspection room. Note that the treatment instrument search unit 38 may be able to obtain, for example, the information related to the treatment instrument existing on the cart by directly communicating with the RFID tag provided in the treatment instrument.

The treatment instrument DB 10 may hold information of treatment instruments of plural types as the treatment instruments suitable to the treatment on the treatment candidate target as illustrated in FIG. 14. In this case, the treatment instrument DB 10 may include information of a treatment instrument (substituted treatment instrument) as a substitute of the treatment instrument candidate decided by the treatment instrument decision unit 35. In such a case, depending on the search information, when the treatment instrument candidate decided by the treatment instrument decision unit 35 does not exist in the inspection room or in the hospital but the substituted treatment instrument exists in the inspection room or in the hospital, the treatment instrument search unit 38 can also obtain information of the substituted treatment instrument as the search result.

The feature, specifications, and performance of the treatment instrument can be searched on the database prepared or provided by the government agencies, industry associations, database service companies or manufacturing companies. The database provides users to search for treatment instrument that meet their conditions based on information on medical departments, disease names, symptoms, treatment candidate target, and equipment used in conjunction with the devices. The information on alternative treatment devices may also be organized and recorded together in the above database.

The treatment instrument DB 10 may be configured to store information through classification of each treatment instrument by image feature of the treatment candidate target. In this case, the treatment instrument decision unit 35 does not necessarily need to decide the treatment instrument candidate by the AI processing or the like in some cases. For example, the treatment instrument decision unit 35 may be configured to obtain information of a classification such as a shape feature or a size feature of the treatment candidate target, and search the treatment instrument search unit 38 for the treatment instrument candidate relevant to the classification.

According to the thus configured embodiment too, an operation similar to the operation of the first embodiment can be achieved. In other words, the treatment instrument candidate is decided in the treatment instrument decision unit 35. The treatment instrument search unit 38 provides information of a location or the like of the decided treatment instrument candidate to the treatment instrument decision unit 35 by searching the treatment instrument DB 10. Accordingly, in the treatment instrument decision unit 35, the guide display for recommending the treatment instrument candidate is displayed.

Furthermore, in the present embodiment, depending on information stored in the treatment instrument DB 10, the information of the classification according to the image feature of the treatment candidate target may be obtained without deciding the treatment instrument candidate in the treatment instrument decision unit 35. The treatment instrument search unit 38 searches the treatment instrument DB 10 based on the information of the treatment instrument candidate from the treatment instrument decision unit 35 or the information of the classification according to the image feature of the treatment candidate target. The treatment instrument search unit 38 supplies the search result to the treatment instrument decision unit 35.

In a case where a treatment instrument candidate exists, for example, on the cart in the inspection room, the treatment instrument decision unit 35 supplies, to the display control unit 37, information indicating that the treatment instrument candidate is suitable to the treatment on the treatment candidate target. The display control unit 37 supplies display data of the guide display for recommending the use of the treatment instrument candidate for the treatment on the detected treatment candidate target to the display unit 41. Thus, the guide display indicating the treatment instrument candidate suitable to the treatment is displayed on the display screen of the display unit 41.

In a case where the treatment instrument candidate does not exist, for example, on the cart in the inspection room but exists in the storage location inside the hospital, the treatment instrument decision unit 35 supplies information on the storage location, the time period for the preparation of the treatment instrument candidate, or the like to the display control unit 37 together with the information indicating the treatment instrument candidate suitable to the treatment on the treatment candidate target. The display control unit 37 supplies, to the display unit 41, the display data of the guide display recommending the use of the treatment instrument candidate for the treatment on the detected treatment candidate target, and indicating the storage location and the time period for the preparation of the treatment instrument candidate, and the like. Thus, the guide display indicating the treatment instrument candidate suitable to the treatment, the storage location, and the like are displayed on the display screen of the display unit 41.

The treatment instrument DB 10 may include the information of the substituted treatment instrument of the treatment instrument candidate decided by the treatment instrument decision unit 35 and the information of the plurality of treatment instruments (substituted treatment instruments) corresponding to the information of the classification according to the image feature of the treatment candidate target. In a case where the treatment instrument candidate decided by the treatment instrument decision unit 35 does not exist in the inspection room or in the hospital, but when the substituted treatment instrument exists in the inspection room or in the hospital, the treatment instrument search unit 38 can obtain the information of the substituted treatment instrument as the search result. In this case, the treatment instrument decision unit 35 may supply the display data of the guide display for recommending the use of the substituted treatment instrument to the display unit 41. In a case where the decided treatment instrument candidate does not initially exist on the cart in the inspection room but in a case where the substituted treatment instrument of the treatment instrument candidate exists on the cart, the treatment instrument decision unit 35 may be configured to supply the guide display indicating the information related to the substituted treatment instrument to the display unit 41 and recommend the use of the substituted treatment instrument.

The treatment instrument decision unit 35 may be configured to select the treatment instrument in the position in closest proximity among a plurality of treatment instrument candidates, for example, the treatment instrument existing on the cart, as the treatment instrument candidate, and display the guide display related to the treatment instrument candidate.

In this manner, in the present embodiment too, an effect similar to the first embodiment can be obtained.

### Third Embodiment

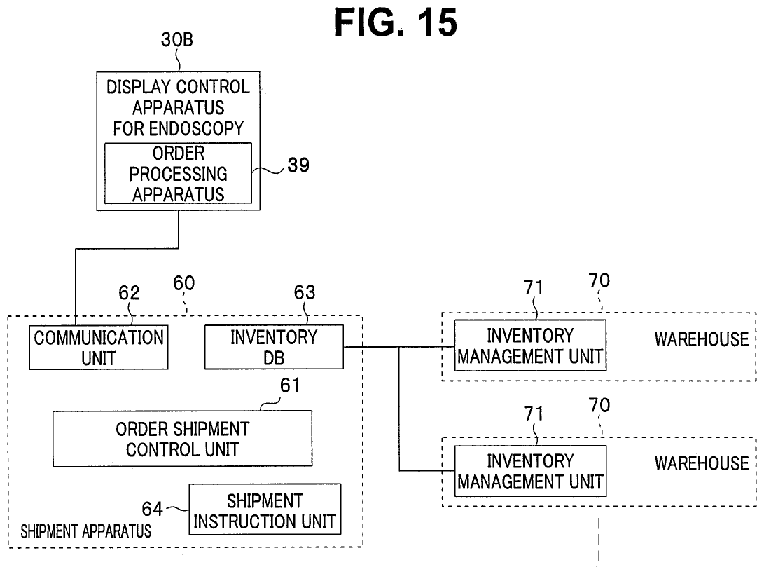

FIG. 15 is a block diagram illustrating a third embodiment. In a case where a treatment instrument does not exist in the inspection room or on the cart, a case where the treatment instrument does not exist in the hospital, or the like, the present embodiment is intended to enable an order for procurement of the treatment instrument. In FIG. 15, a same component as the component in FIG. 1 is assigned with a same reference sign, and a description of the component is not repeated.

In FIG. 15, a display control apparatus for endoscopy 30B is obtained by adding an order processing apparatus 39 to the display control apparatus for endoscopy 30 of FIG. 1 or the display control apparatus for endoscopy 30A of FIG. 13. The order processing apparatus 39 is controlled by the control unit 31 and configured to generate, in a case where a treatment instrument candidate does not exist in the inspection room but exists in the hospital or a case where the treatment instrument candidate does not exist in the hospital, order information for procurement of the treatment instrument candidate and output the order information to a shipment apparatus 60. The order processing apparatus 39 includes a communication interface (I/F) which is not illustrated, and can transmit the order information to a communication unit 62 of the shipment apparatus 60 via a predetermined communication channel such as the Internet.

Note that the shipment apparatus 60 may be arranged in a management department of the hospital or may be arranged in a warehouse or the like outside the hospital. In a case where the shipment apparatus 60 is arranged in the management department of the hospital, a warehouse 70 of FIG. 15 is a warehouse in the hospital. In a case where the shipment apparatus 60 is arranged in the warehouse or the like outside the hospital, the warehouse 70 of FIG. 15 is a warehouse of a manufacturer or the like.

The shipment apparatus 60 includes an order shipment control unit 61, the communication unit 62, an inventory database (DB) 63, and a shipment instruction unit 64. The order shipment control unit 61 is configured to control an entirety of the shipment apparatus 60 in an overall manner. The order shipment control unit 61 may be configured by a processor using a CPU, an FPGA, or the like. The order shipment control unit 61 may control each unit by operating according to a program stored in a memory which is not illustrated, or may realize some or all of functions by a hardware electronic circuit.

The inventory database DB 63 communicates with an inventory management unit 71 provided in each of a plurality of warehouses 70. Each of the warehouses 70 stores a plurality of treatment instruments of various types. The inventory management unit 71 of each of the warehouses 70 manages inventory information related to the inventory of treatment instruments stored in the respective warehouses 70.

The inventory database DB 63 obtains and stores the respective inventory information from the inventory management unit 71 of each of the warehouses 70. By accessing the inventory database DB 63, the order shipment control unit 61 grasps stock statuses of the treatment instruments of the various types. When the order information received by the communication unit 62 is supplied, the order shipment control unit 61 accesses the inventory database DB 63, so that the stock status of the relevant treatment instrument can be checked. In a case where the treatment instrument candidate is in stock, the order shipment control unit 61 instructs the shipment instruction unit 64 to issues an instruction to ship the treatment instrument candidate. The shipment instruction unit 64 is controlled by the order shipment control unit 61, and is configured to issue a shipment instruction of the treatment instrument specified by the order information to the management department of the hospital, an external distribution center, or the like which is not illustrated.

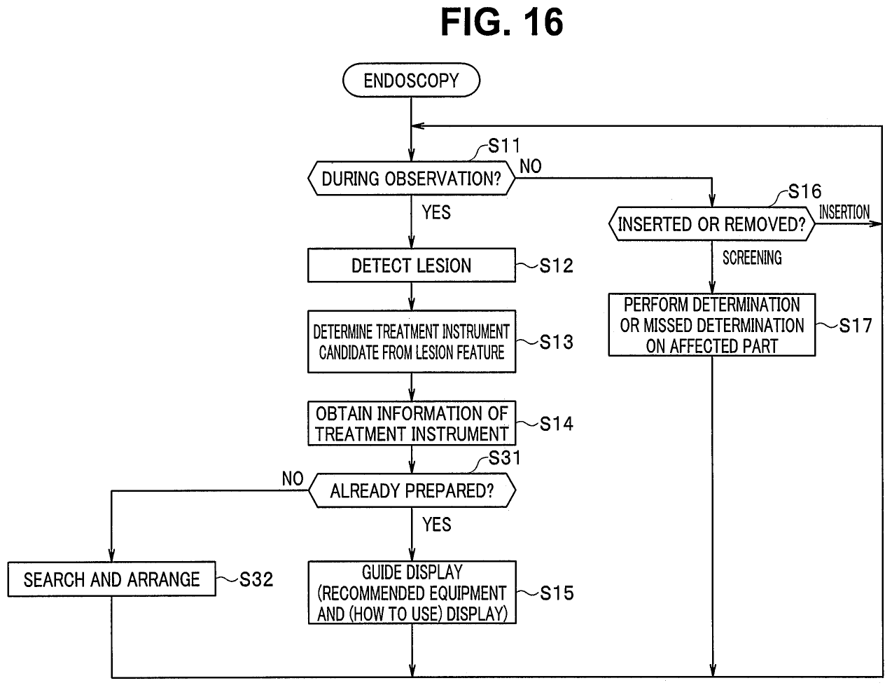

Next, an operation of the thus configured embodiment will be described with reference to FIG. 16. FIG. 16 is a flowchart for describing an operation of the third embodiment. In FIG. 16, a same procedure as the procedure in FIG. 9 is assigned with a same reference sign, and a description of the procedure is not repeated.

In the flow of FIG. 16, a difference from the flow of FIG. 9 is that steps S31 and S32 are added. In S31, it is determined on whether or not the treatment instrument candidate suited to the treatment candidate target is prepared, that is, for example, whether or not the treatment instrument candidate exists on the cart in the inspection room. In S14, the control unit 31 recognizes, by the obtained information of the treatment instrument candidate, for example, whether or not the treatment instrument candidate exists on the cart CA. In a case where the treatment instrument candidate exists on the cart, since the preparation for the treatment is ready (YES in S31), the control unit 31 shifts the processing to S15. In a case where the treatment instrument candidate does not exist on the cart CA, since it is necessary to procure the treatment instrument, and the preparation for the treatment is not ready (NO in S31), the control unit 31 shifts the processing to S32. The control unit 31 controls the order processing apparatus 39 to search for the storage location of the treatment instrument candidate and makes an arrangement for the procurement.

In other words, the order processing apparatus 39 outputs the order information of the treatment instrument candidate to the shipment apparatus 60. The order information is supplied to the order shipment control unit 61. The order shipment control unit 61 accesses the inventory database DB 63 to check a stock status, that is, an availability of stock, receiving and shipping date and time, or the like, with regard to the treatment instrument candidate specified by inventory check information. In a case where the treatment instrument candidate is in stock, the order shipment control unit 61 instructs the shipment instruction unit 64 to ship the treatment instrument specified by the order information.

In this manner, according to the present embodiment, in a case where the treatment instrument candidate for treating the treatment candidate target does not exist in the inspection room or the like, the treatment instrument candidate can easily be ordered.

The present disclosure is not directly limited to each of the embodiments, and in an implementation stage, a component can be modified to be embodied without departing from the gist. Various aspects can be created by appropriately combining a plurality of components disclosed in each of the embodiments. For example, some components may be deleted from all the components illustrated in the embodiments. Furthermore, components across different embodiments may appropriately be combined.

For example, herein, the treatment instrument has been described in a situation in which gastrointestinal endoscopy, treatment, or the like is assumed in particular. However, the technology can be applied in a case where upon inspection using a specific image of a target object, a treatment is involved on the target object needed, and image pickup inspection equipment and system can be assumed. The technology is not intended to be limited to an endoscope or medical equipment.

Among the technologies described herein, many of the controls and functions mainly described in the flowcharts can be set by a program, and the above mentioned controls and functions can be realized by reading and executing the program by a computer. The program can be recorded or stored as a computer program product in whole or in part on a portable medium such as a non-volatile memory like a flexible disk or a CD-ROM, or a storage medium such as a hard disk or a volatile memory. The program can be distributed or provided at the time of product shipment or via the portable medium or a communication line. A user downloads the program via the communication network and installs the program on the computer, or installs the program on the computer from the recording medium, so that the display control apparatus for endoscopy according to the present embodiment can easily be realized. A non-transitory computer readable storage medium stores an endoscopic control program configured for execution by a computer system having one or more processors and memory. The transitory computer readable storage medium comprises detecting a treatment candidate of an image feature based on a picked-up image obtained from an endoscope during an endoscopy, determining a treatment instrument based on the image feature of the treatment candidate, and generating guide information. The guide information includes information related to the treatment instrument.

### Example 1

A display control apparatus for endoscopy, comprising:
a processor is configured to:
detects a treatment candidate target of a specific image feature based on a picked-up image obtained upon endoscopy;
determines a corresponding treatment instrument candidate based on the image feature of the treatment candidate target; and
displays a guide display including information of the treatment instrument candidate.

### Example 2

The display control apparatus for endoscopy according to Example 1, wherein
the processor is configured to display the guide display including information on a location of the treatment instrument candidate.

### Example 3

The display control apparatus for endoscopy according to Example 1, wherein
the processor is configured to display the guide display indicating that the treatment instrument candidate exists on a cart on which a treatment instrument is placed upon the endoscopy.

### Example 4

The display control apparatus for endoscopy according to Example 3, wherein
in a case where the treatment instrument candidate does not exist on the cart but a substituted treatment instrument that is a substitute of the treatment instrument candidate exists on the cart, the processor is configured to display information of the substituted treatment instrument as the guide display.

### Example 5

The display control apparatus for endoscopy according to Example 1, wherein
the processor is configured to determine the treatment instrument candidate by using, with respect to a series of frames of an endoscopic image obtained by the endoscopy, an inference model obtained by leaning training data obtained by annotating a treatment instrument appearing in the endoscopic image.

### Example 6

The display control apparatus for endoscopy according to Example 1, wherein
the processor is configured to:
calculate a treatment time period spent to treat a lesion part based on an endoscopic image obtained by the endoscopy, and
display information of a degree of difficulty analogized based on the treatment time period upon display of the treatment instrument candidate.

### Example 7

The display control apparatus for endoscopy according to Example 1, further comprising:
a communication apparatus configured to wirelessly communicate with an electronic tag provided in the treatment instrument, wherein
the processor checks a presence of a treatment instrument corresponding to the treatment instrument candidate by communication between the communication apparatus and the electronic tag.

### Example 8

A display control method for endoscopy, comprising:
detecting a treatment candidate target of a specific image feature based on a picked-up image obtained upon endoscopy;
determining a corresponding treatment instrument candidate based on the image feature of the treatment candidate target; and
displaying a guide display including information of the treatment instrument candidate on display equipment configured to display the picked-up image.

### Example 9

A non-transitory recording medium recording a display control program for endoscopy for causing a computer to execute a procedure comprising:
detecting a treatment candidate target of a specific image feature based on a picked-up image obtained upon endoscopy;
determining a corresponding treatment instrument candidate based on the image feature of the treatment candidate target; and
displaying a guide display including information of the treatment instrument candidate on display equipment configured to display the picked-up image.

### Example 10

A medical system comprising:
an endoscope;
display equipment; and
a display control apparatus for endoscopy including a processor, wherein the processor detects a treatment candidate target of a specific image feature based on a picked-up image obtained upon endoscopy, determines a corresponding treatment instrument candidate based on the image feature of the treatment candidate target, and displays a guide display including information of the treatment instrument candidate on the display equipment configured to display the picked-up image.

### Example 11

A training data creation apparatus comprising a processor, wherein
the processor is configured to:
determine a timing at which a treatment on a specific target object is performed by a plurality of image frames obtained from an image pickup device of an endoscope apparatus,
determine a type of a treatment instrument based on a feature of an image appearing in a specific direction in the image frames at timings before and after the timing at which the treatment on the specific target object is performed, and
annotate information of the treatment instrument with respect to the image frames.

### Example 12

A display control method for endoscopy, comprising:
detecting a treatment candidate target of a specific image feature based on a picked-up image obtained upon endoscopy;
in order to infer a corresponding treatment instrument candidate based on the image feature of the treatment candidate target, from among continuous frames configuring a plurality of movies upon endoscopy which are obtained in advance, detecting frames in which a treatment instrument which appears in respective endoscopic images appears, and determining the treatment instrument candidate by using a result of an inference in which an image frame including the treatment candidate target image of the specific image feature obtained upon the endoscopy is inputted to an inference model obtained by learning a relationship between a treated treatment candidate target image and the treatment instrument used upon the endoscopy by using a training data group obtained by annotating an image part corresponding to the treatment instrument from the frames in which the treatment instrument appears; and
displaying a guide display including information of the treatment instrument candidate which is inferred on display equipment configured to display the picked-up image.

### Example 13

A non-transitory computer readable storage medium storing an endoscopic control program according to an aspect of the present disclosure records the endoscopic control program that causes a computer to execute a procedure including detecting a treatment target of an image feature based on a picked-up image obtained from an endoscope during endoscopy, determining a treatment instrument based on the image feature of the treatment candidate, and generating a guide information, the guide information includes information related to the treatment instrument.

### Example 14

A training data creation apparatus according to an aspect of the present disclosure comprises a processor, the processor is configured to determine a time duration for a treatment on a target object based on a plurality of image frames obtained from an image pickup device of an endoscope apparatus, determine a type of a treatment instrument based on a feature of an image appearing in a specific direction in the plurality of image frames at a first time and at a second time, and annotates information of the type of treatment instrument with respect to each of the plurality of image frames. The first time is before the treatment on the target object is performed and the second time is after the treatment on the target object is performed.

What is claimed is:

1. An endoscopic control apparatus, comprising:
one or more processors configured to:
process image matching with respect to a first image and a second image to determine an image in the first image and the second image, the first image obtained from an endoscope during an endoscopy, the image feature comprises information related to common pixels among the first image and the second image;
detect a treatment target based on the image feature obtained from the image matching,
determine a treatment instrument based on the detected treatment target, and
display guide information,
wherein the guide information includes information related to the treatment instrument.

2. The endoscopic control apparatus according to claim 1, wherein the one or more processors is configured to:
process one or more color adjustment processing, matrix conversion processing, noise removal processing to the image.

3. The endoscopic control apparatus according to claim 1, wherein the second image is obtained from the endoscope during the endoscopy, and
the image feature includes information related to pixels corresponding a common feature between the first image and the second image, the common feature is obtained from image matching.

4. The endoscopic control apparatus according to claim 1, wherein the second image is a template image, and
the image feature includes information related to pixels corresponding a common feature between the first image and the second image, the common feature is obtained from image matching.

5. The endoscopic control apparatus according to claim 1, wherein the image feature includes a blurring amount of a distal end of the endoscope.

6. The endoscopic control apparatus according to claim 1, wherein the guide information includes a location of the treatment instrument.

7. The endoscopic control apparatus according to claim 1, wherein the one or more processors is configured to:
determine whether the treatment instrument is present on a treatment instrument cart; and
in response to the determining that the treatment instrument is present on the treatment instrument cart, output the guide information including the treatment instrument presented on the treatment instrument cart.

8. The endoscopic control apparatus according to claim 4, wherein the one or more processors is configured to:
in response to the determining that the treatment instrument is not present on the treatment instrument cart, determine whether a substitute treatment instrument is present on the treatment instrument cart, and
the guide information includes information related to the substitute treatment instrument.

9. The endoscopic control apparatus according to claim 1, wherein the one or more processors is configured to determine the treatment instrument using an inference model, and
wherein the inference model is obtained from learning training data obtained by annotating a treatment instrument appearing in an endoscopic image obtained from the endoscope during the endoscopy.

10. The endoscopic control apparatus according to claim 1, wherein the one or more processors is configured to:
determine the treatment instrument based on the detected treatment target and a database reference.

11. The endoscopic control apparatus according to claim 1, wherein the one or more processors is further configured to:
calculate a treatment time period for treating a lesion part based on an endoscopic image obtained from the endoscope during the endoscopy; and
determine a degree of difficulty based on the treatment time period and the treatment instrument, and
wherein the guide information includes the degree of difficulty.

12. The endoscopic control apparatus according to claim 1, further comprising:
a communication apparatus configured to wirelessly communicate with an electronic tag provided in the treatment instrument,
wherein the one or more processors is configured to check for a presence of the treatment instrument by communication between the communication apparatus and the electronic tag.

13. The endoscopic control apparatus according to claim 1, wherein the image feature indicates one or more of a location of the treatment target, a shape of the treatment target, a size of the treatment target, a color of the treatment target, an edge of the treatment target, a focused area of the treatment target, concavity and convexity about the treatment target, a blood pattern about the treatment target, a blood arrangement about the treatment target or a distance between the endoscope and the treatment target.

14. The endoscopic control apparatus according to claim 1, wherein the treatment instrument comprises one of a snare, a biopsy forceps, a grasping forceps, a basket, a high frequency a knife or a syringe needle.

15. An endoscope system comprising:
the endoscopic control apparatus according to claim 1.

16. The endoscope system according to claim 15, further comprising:
display equipment.

17. A medical system, comprising:
the endoscope system according to claim 15; and
display equipment.

18. An endoscopic control method, comprising:
processing image matching with respect to a first image and a second image to determine an image feature in the first image and the second image, the first image obtained from an endoscope during an endoscopy, the image feature comprises information related to common pixels among the first image and the second image;
detecting a treatment target of the image feature obtained from the image matching;
determining a treatment instrument based on the image feature of the treatment target; and
displaying guide information,
wherein the guide information includes information related to the treatment instrument.

19. An endoscopic control method, comprising:
detecting a treatment target of a first image feature from first images obtained from an image pick-up device of an endoscope during a first endoscopy;
determine a second image feature based on the first image feature, the second image feature is obtained from a second images during a second endoscopy being conducted prior to the first endoscopy;
determining a treatment instrument based on the second image feature, the treatment instrument being included in third image subsequent to the second image; and
determining a treatment instrument candidate corresponding to the treatment instrument; and
displaying guide information including information related to the treatment instrument candidate.

20. The endoscopic control method according to claim 19, wherein the determining that the treatment instrument comprises:
referencing a database storing the second image feature associated with the treatment instrument.
